(19) United States
(12) Patent Application Publication
(10) Pub. No.: US 2006/0019565 A1
(43) Pub. Date: Jan. 26, 2006

(54) Method for delivering biologically active substnces

(76) Inventors: Stefan Koller, Ramlinsburg (CH); Veronique Hall-Goulle, Dornach (CH)

Correspondence Address: CIBA SPECIALTY CHEMICALS CORPORATION;PATENT DEPARTMENT, 540 WHITE PLAINS RD, P O BOX 2005, TARRYTOWN, NY, 10591-9005, US

(21) Appl. No.: 10/524,007

(22) PCT Filed: Aug. 19, 2003
(86) PCT No.: PCT/EP03/09164

§ 371 Date: Feb. 8, 2005

(30) Foreign Application Priority Data
Aug. 27, 2002 (EP) 02405723.4

(51) Int. Cl. B32B 27/04; C07D 263/34; C07D 233/22; C07D 213/56

(52) U.S. Cl. 442/123; 546/316; 548/333.5; 548/236

(57) ABSTRACT

The present application relates to a method for the controlled and/or slow release of a biologically active hydroxyl group containing-substance on a substrate which comprises reacting said hydroxyl group containing substance subsequently with a halogen-substituted aliphatic carboxylic acid halide and either a diamine containing at least one tertiary amino group or a heterocyclic aromatic amine, applying the thus obtained water-soluble ester to the substrate and finally hydrolysing the ester on the substrate.

The present invention relates to a method for releasing a biologically active hydroxyl group containing substance on a substrate and to an aqueous solution containing the reaction product of a biologically active hydroxyl group containing substance, a halogen-substituted aliphatic carboxylic acid halide and a tertiary diamine or a heterocyclic aromatic amine.

U.S. Pat. No. 4,083,847 describes transiently water-soluble disperse dyes that contain a group which can be removed under dying conditions and which carries at least one water-solubilising group. Addition of large amounts of dispersing agents and stabilizers can thus be avoided.

It has now unexpectedly been found that this principle can be used for the controlled release of biologically active compounds of any kind on various types of substrates by applying a blocked compound in the form of an aqueous solution and later deblocking under hydrolytic conditions.

The present invention relates to a method for the controlled release of a biologically active hydroxyl group containing substance on a substrate, which comprises reacting said hydroxyl group containing substance subsequently with a halogen-substituted aliphatic carboxylic acid halide and either a diamine containing at least one tertiary amino group or a heterocyclic aromatic amine, applying the thus obtained water-soluble ester to the substrate and finally hydrolysing the ester on the substrate.

Suitable biologically active hydroxyl group containing substances are any types of drugs, for example pain relief agents like paracetamol and acetylsalicylic acid, vitamins like ascorbic acid, hormones like testosterone and estradiol.

Plant protective agents like herbicides, fungicides, insecticides and bactericides can likewise be used in the method according to the invention.

Other suitable biologically active substances are flavouring agents, like menthol and cosmetics.

Other preferred biologically active substances which can be used in the claimed process are insecticides or antimicrobials, like triclosan.

In the first step of the claimed process the hydroxyl group containing substance R—OH is reacted with a halogen-substituted aliphatic carboxylic acid halide thus yielding the corresponding halogen-substituted acid ester.

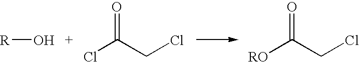

Afterwards a water-soluble ammonium salt is prepared by reaction of the halogen-substituted ester with a diamine containing at least one tertiary amino group or a heterocyclic aromatic amine.

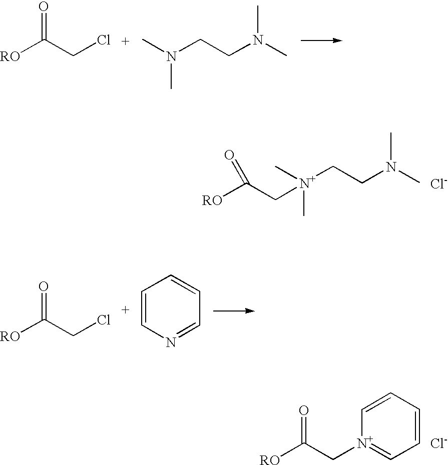

Preferred diamines containing at least one tertiary amino group are the diamines of general formula R₁R₂N-A-NR₃R₄ wherein R₁ and R₂ are independently C₁-C₇ alkyl, R₃ and R₄ are Independently H or C₁-C₇ alkyl and A is a C₁-C₇ linear or branched alkyl chain.

Examples for suitable diamines R₁R₂N-A-NR₃R₄ are 1,2-bis(dimethylamino)ethane, 1,3-bis(dimethylamino)propane, 1,2-bis(dimethylamino)propane, 1,4-bis(dimethylamino)butane, 1,3-bis(dimethylamino)butane, 2,3-bis(dimethylamino)butane, 1,5-bis(dimethylamino)-2-pentene, 1,5-bis(dimethylamino)pentane, 1,6-bis(dimethylamino)hexane, 1,7-bis(dimethylamino)heptane, 1, 1,2-bis(diethylamino)ethane, 1,3-bis(diethylamino)propane, 1,2-bis(diethylamino)propane, 1,4-bis(diethylamino)butane, 1,3-bis(diethylamino)butane, 2,3-bis(diethylamino)butane, 1,5-bis(diethylamino)-2-pentene, 1,5-bis(diethylamino)pentane, 1,6-bis(diethylamino)hexane, 1,7-bis(diethylamino)heptane, 1,4-bis(dimethylamino)-1,3-butadiene and 1-dimethylamino-2-methyloctylamino-ethane.

1,2-bis(dimethylamino)ethane is the preferred diamine.

Heterocyclic aromatic amines that can be applied in the method according to the invention may be pyrroles, imidazoles, oxazoles, pyridines, 1,2-, 1,3- and 1.4-diazines, 1,2-, 1,3- and 1.4-triazines as well as benzopyrroles, benzimidazoles, quinolines, isoquinolines and bipyridyls.

The aforementioned heterocyclic aromatic amines may be unsubstituted or can be substituted by one or more halogen atoms, cyano groups, alkyl groups, alkoxy groups or dialkylamino groups.

Preferably, the heterocyclic aromatic amine is an unsubstituted or substituted pyridine, bipyridyl, imidazole or oxazole.

Pyridine, 4-dimethylaminopyridine, 4-methoxypyridine, 4-cyanopyridine and 4,4′-bipyridyl are particularly preferred.

The blocked compounds exhibit a high solubility in cold water and accordingly can be applied as aqueous solutions to a variety of substrates like wood, plastics, paper and textile materials.

Preferably, the method according to the invention is used for furnishing paper or textile fabrics.

Suitable substrates are, for example, materials like polyacrylonitril and copolymers of acrylonitrile and other vinyl compounds, e.g. acrylic esters, acrylic amides, vinyl pyridine, vinyl chloride or vinylidene chloride, copolymers of dicyanoethylene and vinyl acetate as well as of acrylonitrile block copolymers, polyurethanes, synthetic polyamides, e.g. poly(hexamethylene adipic acid amide) or polyamide 66, poly(ε-caprolactame) or polyamide 6, poly(hexamethylenesebacic amide) or polyamide 610 and poly(11-aminoundecanoic acid) or polyamide 11, cellulose triacetate and cellulose 2½ actetate, polyesters, and in particular all cellulose based substrates like cotton and viscose, and mixed fibers containing cellulose. These materials can be in the most widely differing processed forms, for example spun yarns, knitted fabrics, woven fabrics, yarns or fibres.

The process according to the invention is easy to operate and can be carried out by the conventional methods known in the art of textile dying, for example the exhaust process or the padding process.

This application process of the ester compound is usually carried out at elevated temperature, for example at 60° C. to 130° C., if appropriate under pressure, in a slightly acidic, slightly alkaline or neutral bath at a pH of 3 to 8, preferably 4 to 7 and in particular 4.5 to 6. Buffer systems containing, for example, phosphates or carboxylates may be added to the bath.

An aqueous solution containing the reaction product of a biologically active hydroxyl group containing substance, a halogen-substituted aliphatic carboxylic acid halide and either a diamine containing at least one tertiary amino group or a heterocyclic aromatic amine is a further object of the invention.

After the treatment with the aqueous solution of the ester compound a slow release of the biologically active hydroxyl compound on the substrate starts through hydrolysis. The velocity of this process can easily be controlled through pH and/or temperature variations.

The following examples illustrate the invention.

### I. SYNTHESIS EXAMPLES

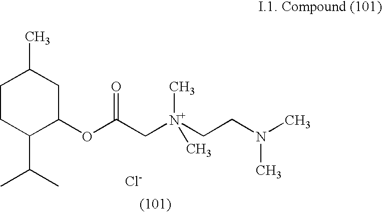

Menthol is first reacted with chloroacetyl chloride in methyl ethyl ketone/pyridine and subsequently with N,N,N′N′-tetramethylethylene diamine according to conventional methods to yield the menthol derivative (101).

NMR (D₂O) δ 0.72 (d, 3H, —CH₃), 0.81-0.87 (m, 7H), 1.02-1.13 (m, 2H), 1.35-1.55 (m, 2H), 1.57-1.80 (m, 3H), 1.90-2.00 (m, 1H), 2.27 (s, 6H, —N(CH₃)₂), 2.75-2.92 (m, 2H, —CH₂—N), 3.26 (d, 6H, +N(CH₃)₂), 3.60-3.80 (m, 2H, +N—CH₂—), 4.65 (s, 2H, —(C=0)—CH₂—N+), 4.81 (s, 1H, —CH—O).

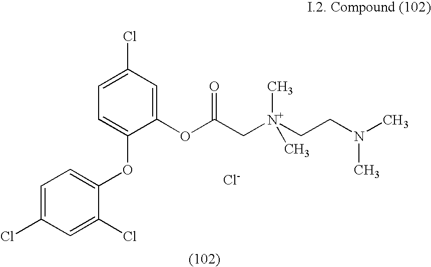

Triclosan is first reacted with chloroacetyl chloride in methyl ethyl ketone/pyridine and subsequently with N,N,N′N′-tetramethylethylene diamine according to conventional methods to yield the triclosan derivative (102).

NMR (CDCl₃) δ 2.22 (s, 6H, —N(CH₃)₂), 2.76 (m, 2H, —CH₂—N), 3.70 (s, 6H, +N(CH₃)₂), 3.98 (m, 2H, +N—CH₂—), 5.25 (s, 2H, —(C=0)CH₂—N+), 6.71 (d, 1H, Ar—H), 6.94 (d, 1H, Ar—H), 7.16-7.22 (m, 2H, Ar—H), 7.29 (d, 1H, Ar—H), 7.46 (d, 1H, Ar—H).

In the same way compounds (103) to (129) are prepared according to conventional methods:

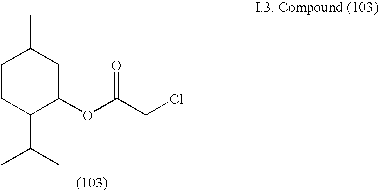

NMR (DMSO-d₆) δ 0.73 (d, 3H, —CH₃), 0.88 (m, 7H), 0.90-1.15 (m, 2H), 1.25-1-58 (m, 2H), 1.60-1.70 (m, 2H), 1.80-1.95 (m, 2H), 4.31 (m, 2H, —(C═O)—CH₂—Cl), 4.65 (m, 1H, —CH—O).

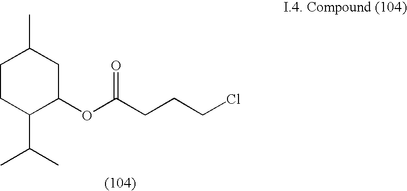

NMR DMSO-d₆ δ 0.72 (d, 3H, —CH₃), 0.87 (m, 7H), 0.90-1.15 (m, 2H), 1.25-1-55 (m, 2H), 1.55-1.70 (m, 2H), 1.70-1.92 (m, 2H), 1.97 (q, 2H, —CH₂—), 2.43 (t, 2H, —CH₂—Cl), 3.63 (t, 2H, —(C═O)—CH₂—), 4.59 (m, 1H, —CH—O).

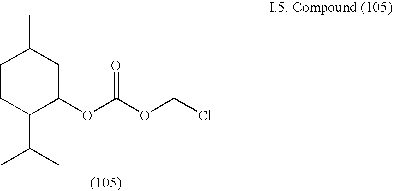

NMR (CDCl₃) δ 0.78 (d, 3H, —CH₃), 0.91 (m, 7H), 0.99-1.15 (m, 2H), 1.35-1-55 (m, 2H), 1.60-1.74 (m, 2H), 1.86-1.98 (m, 1H), 2.04-2.14 (m, 1H), 5.58 (m, 1H, —CH—O), 5.70 (m, 2H, —O—CH₂—Cl).

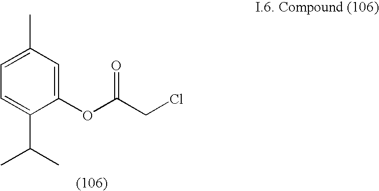

NMR (DMSO-d₆) δ 1.14 (d, 6H, —CH₃), 2.27 (s, 3H, —CH₃). 3.00 (q, 1H, —CH), 4.69 (s, 2H, —(C═O)—CH₂—Cl), 6.90 (s, 1H. Ar—H), 7.05 (d, 1H, Ar—H), 7.24 (d, 1H, Ar—H).

Analysis: C₁₂H₁₅ClO₂

Calculated: C 63.58, H 6.67, O 14.11, Cl 15.64. Found: C 64.08, H 7.01, O 13.98, Cl 15.0.

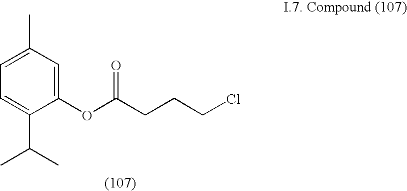

NMR (DMSO-d₆) δ 1.13 (d, 6H, —CH₃), 2.10 (q, 2H, —CH₂—), 2.26 (s, 3H, —CH₃), 2.76 (t, 2H, —CH₂—Cl), 2.92 (q, 1H, —CH), 3.72 (t, 2H, —(C═O)—CH₂—), 6.84 (s, 1H, Ar—H), 7.01 (d, 1H, Ar—H), 7.21 (d, 1H, Ar—H).

Analysis: C₁₄H₁₉ClO₂

Calculated: C 66.01, H 7.52, O 12.56, Cl 13.92. Found: C 65.97, H 7.57, O 12.51, Cl 13.9.

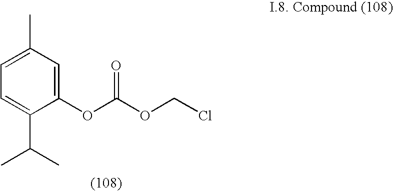

NMR (CDCl₃) δ 1.26-1.28 (d, 6H, —CH₃), 2.38 (s, 3H, —CH₃), 3.05-3.18 (m, 1H, —CH), 5.85 (s, 2H, O—CH₂—Cl), 6.94 (s, 1H, Ar—H), 7.10 (d, 1H, Ar—H), 7.26 (d, 1H, Ar—H).

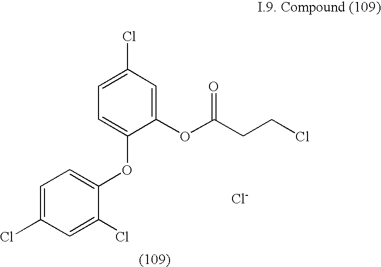

NMR (DMSO-d₆) δ 3.03 (t, 2H, —CH₂—Cl), 3.79 (t, 2H, —(C═O)—CH₂—), 6.95 (d, 1H, Ar—H), 7.08 (d, 1H, Ar—H), 7.30-7.39 (m, 2H, Ar—H), 7.46 (s, 1H, Ar—H), 7.71 (s, 1H, Ar—H).

Analysis: C₁₅H₁₀Cl₄O₃

Calculated: C 47.41, H 2.65, O 12.63, Cl 37.31. Found: C 47.62, H 2.86, O 12.69, Cl 37.0.

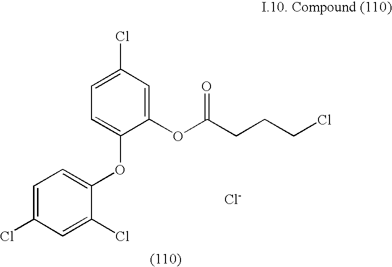

NMR (DMSO-d₆) δ 1.96 (q, 2H, —CH₂—), 2.62 (t, 2H, —CH₂—Cl), 3.62 (t, 2H, —(C═O)—CH₂—), 6.92 (d, 1H, Ar—H), 7.09 (d, 1H, Ar—H), 7.31-7.38 (m, 2H, Ar—H), 7.50 (s, 1H, Ar—H), 7.71 (s, 1H, Ar—H).

Analysis: C₁₆H₁₂Cl₄O₃

Calculated: C 48.44, H 3.07, O 12.18, Cl 35.99. Found: C 48.70, H 3.09, O 12.48, Cl 36.5.

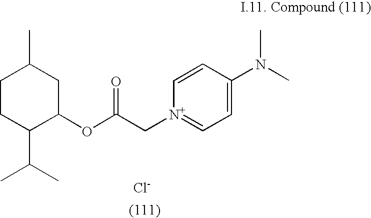

NMR (DMSO-d₆) δ 0.72 (d, 3H, —CH₃), 0.86-0.89 (m, 7H), 100 (m, 2H), 1.24-1.40 (m, 2H), 1.50-1.70 (m, 2H), 1.80-2.00 (m, 2H), 3.29 (s, 6H, N(CH₃)₂), 4.65 (m, 1H, —CH—O), 5.19 (m, 2H, —(C=0)—CH₂—N+), 7.07 (d, 2H, Ar—H), 8.22 (d, 2H, Ar—H).

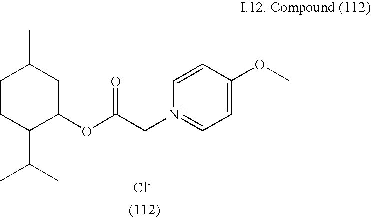

NMR (DMSO-d₆) δ 0.72 (d, 3H, —CH₃), 0.88-0.90 (m, 7H), 1.00-1.08 (m, 2H), 1.28-1.55 (m, 2H), 1.55-1.70 (m, 2H), 1.80-2.10 (m, 2H), 4.12 (s, 3H, O—CH₃). 4.68 (m, 1H, —CH—O), 5.50 (m, 2H, —(C=0)—CH₂—N+), 7.69 (d, 2H, Ar—H), 8.85 (d, 2H, Ar—H).

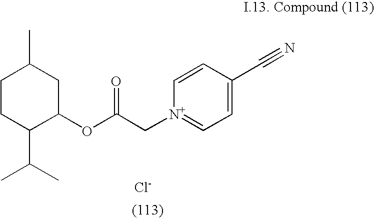

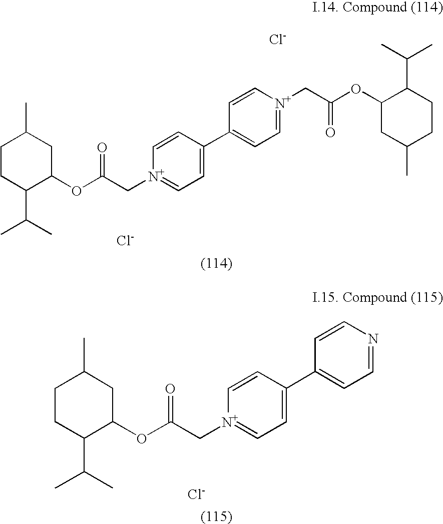

NMR (CDCl₃) δ 0.74 (d, 3H, —CH₃), 0.90 (m, 7H), 0.92-1.18 (m, 2H), 1.30-1.53 (m, 2H), 1.60-1.75 (m, 2H), 1.76-1.92 (m, 1H), 1.94-2.10 (m, 1H), 4.78 (m, 1H, —CH—O), 6.27 (m, 2H, —(C═O)—CH₂—N+), 7.67 (d, 2H, Ar—H), 8.26 (d, 2H, Ar—H), 8.85 (d, 2H, Ar—H), 9.46 (d, 2H, Ar—H).

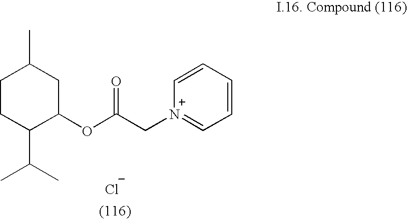

NMR (CDCl₃) δ 0.72 (d, 3H, —CH₃), 0.88 (m, 7H), 0.92-1.15 (m, 2H), 1.28-1.55 (m, 2H), 1.58-1.70 (m, 2H), 1.72-1.92 (m, 1H, —CH), 1.94-2.08 (m, 1H, —CH), 4.75 (m, 1H, —CH—O), 6.29 (m, 2H, —(C═O)—CH₂—), 8.04 (t, 2H, Ar—H), 8.50 (t, 1H, Ar—H), 9.45 (d, 2H, Ar—H).

Analysis: C₁₇H₂₆NO₂Cl

Calculated: C, 65.48; H, 8.40; N, 4.49, O 10.26, Cl 11.37. Found: C, 65.40; H, 8.47; N, 4.46, O 10.27, Cl 11.5.

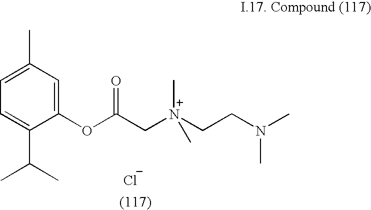

NMR (CDCl₃) δ 1.17 (d, 6H, —CH₃), 2.28 (s, 6H, N(CH₃)₂), 2.29 (s, 3H, —CH₃), 2.82-2.87 (m, 2H, —CH₂—), 2.96 (q, 1H, —CH), 3.81 (s, 6H, +N(CH₃)₂), 4.05-4.10 (m, 2H, +N—CH₂—), 5.43 (s, 2H, —(C═O)—CH₂-N+), 6.80 (s, 1H, Ar—H), 7.03 (d, 1H, Ar—H), 7.18 (d, 1H, Ar—H).

Analysis: C₁₈H₃₄N₂O₂Cl

Calculated: C, 62.50; H, 9.91; N, 8.10, O 9.25, Cl 10.25. Found: C, 62.49; H, 9.13; N, 8.10, O 9.45, Cl 10.5.

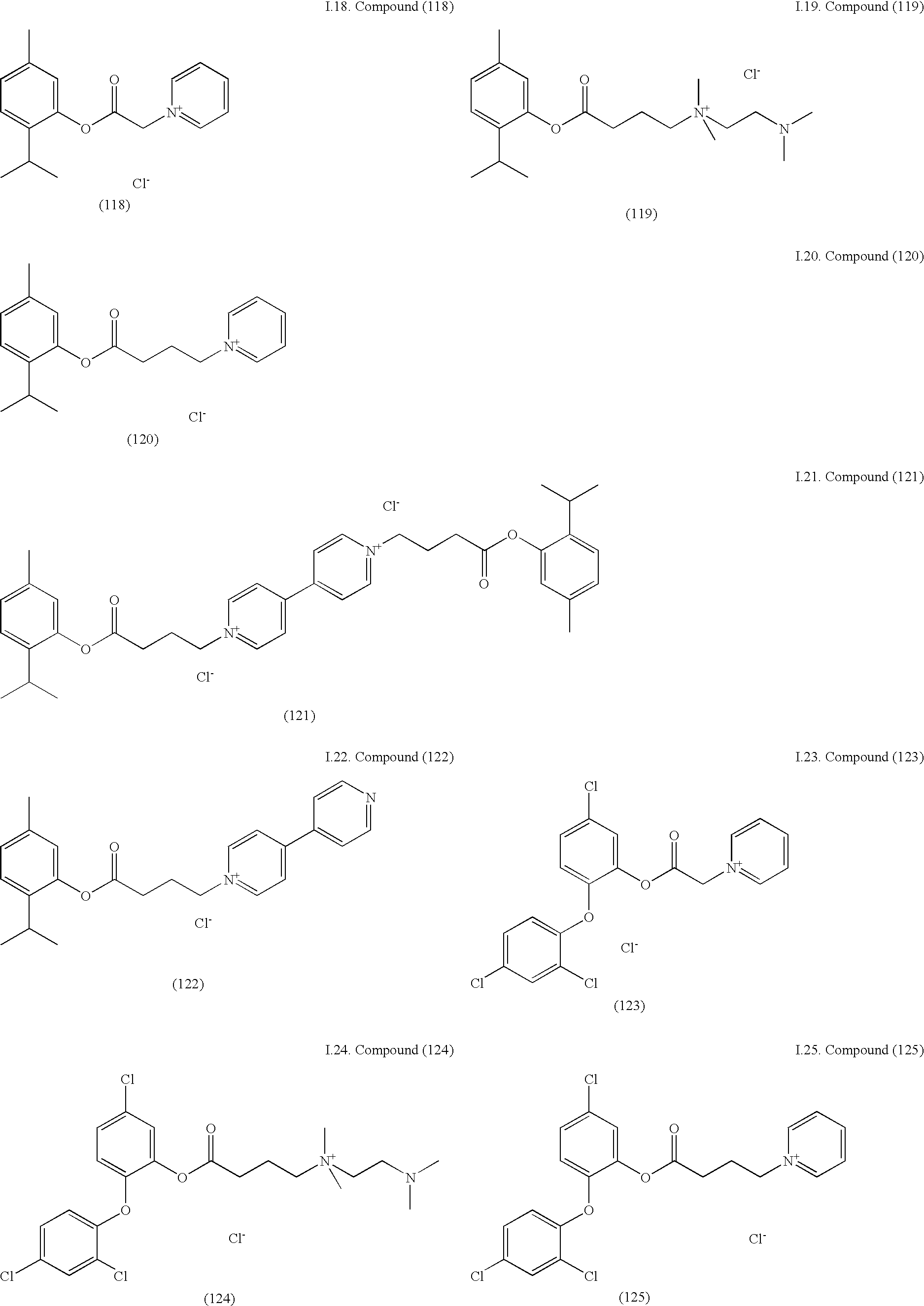

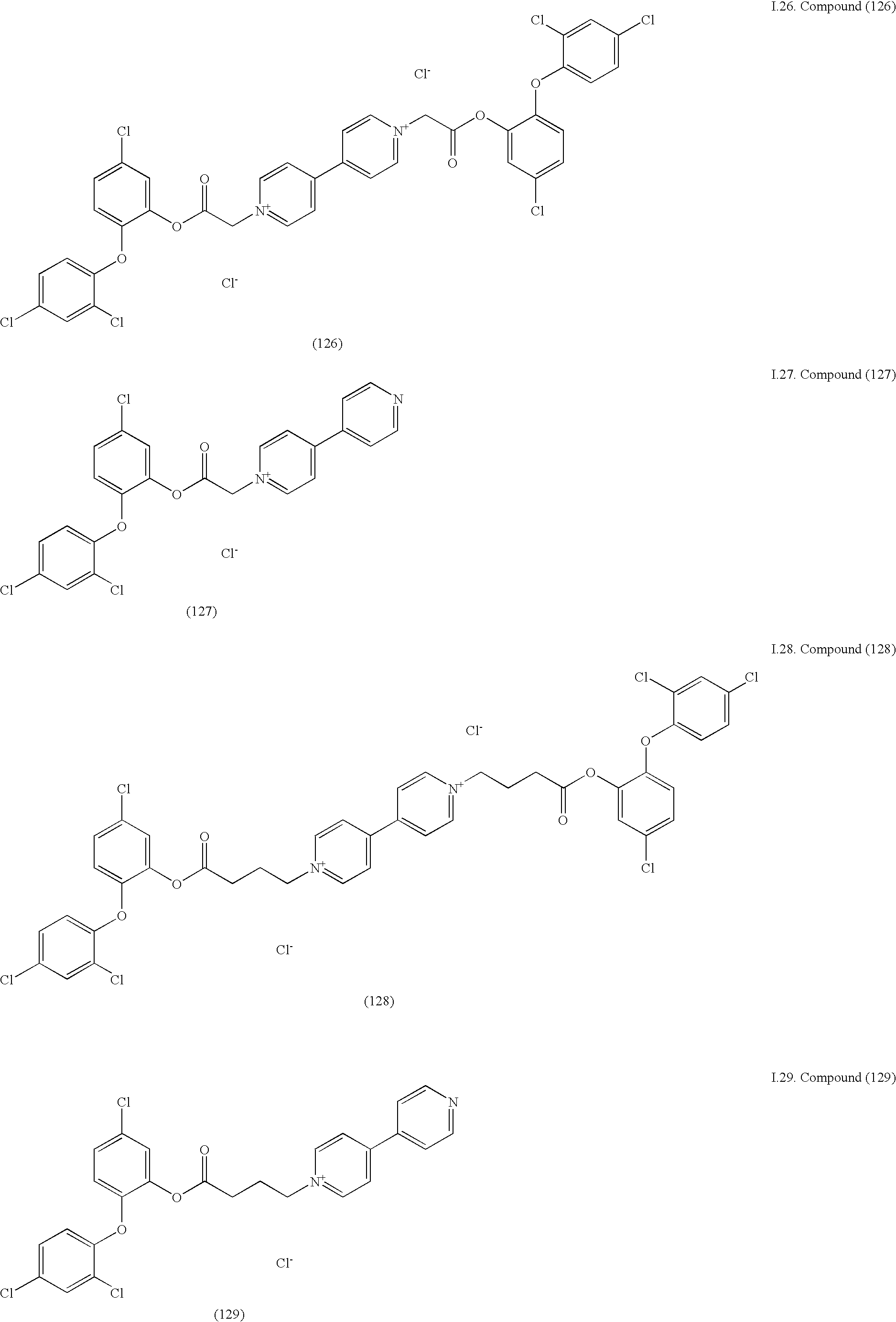

### II. APPLICATION EXAMPLES

II.1 A sample of 15 g polyacrylonitrile (PAN) fabric (Dralon 5-4301), pretreated with a commercial wetting agent (TINOVETIN® JU, supplied by Ciba Specialty Chemicals) at 60° C. during 10 min and rinsed with cold water, is fixed on a support material and in an exhaust dyeing machine of type Ahiba treated with the following composition:
7.5 ml aqueous Na₂SO₄ solution (100 g/l)
2.25 ml aqueous sodium acetate solution (100 g/A)
2.25 ml 80% acetic acid
233.5 ml water
3.0 ml cationic retarder (TINEGAL® MR, Ciba Specialty Chemicals) (100 g/l)
1.5 ml aqueous solution of compound of formula (101) (100 g/l)

The temperature is kept at 98° C. for 20 min; the pH of the bath after cooling to room temperature is 4.7. The sample of PAN fabric is rinsed with cold water and subsequently dried in the air. The resulting PAN fabric contains the latent menthol. Menthol is gradually regenerated from this fabric; the velocity of menthol release is controlled by pH.

II.2 A PAN fabric (14.9 g) is treated as described in Example 11.1 with the following composition:
7.5 ml aqueous Na₂SO₄ solution (100 g/l)
2.25 ml aqueous sodium acetate solution (100 g/l)
2.25 ml 80% acetic acid
232.75 ml water
1.5 ml cationic retarder (TINEGAL® MR, Ciba Specialty Chemicals) (100 g/l)
3.75 ml aqueous solution of compound of formula (101) (100 g/l)

The temperature is kept at 98° C. for 20 min; the pH of the bath after cooling to room temperature is 4.7. The sample of PAN fabric is rinsed with cold water and subsequently dried in the air. The resulting PAN fabric contains the latent menthol. Menthol is gradually regenerated from this fabric; the velocity of menthol release is controlled by pH.

II.3 A PAN fabric (15 g) is treated as described in Example II.1 with the following composition:
7.5 ml aqueous Na₂SO₄ solution (100 g/l)
2.25 ml aqueous sodium acetate solution (100 g/l)
2.25 ml 80% acetic acid
230.5 ml water
7.5 ml aqueous solution of compound of formula (101) (100 g/l)

The temperature is kept at 98° C. for 20 min; the pH of the bath after cooling to room temperature is 5.0. The sample of PAN fabric is rinsed with cold water and subsequently dried in the air. The resulting PAN fabric contains the latent menthol. Menthol is gradually regenerated from this fabric; the velocity of menthol release is controlled by pH.

II.4 A PAN fabric (14.8 g) is treated as described in Example II.1 with the following composition:
2.25 ml aqueous sodium acetate solution (100 g/l)
2.25 ml 80% acetic acid
220 ml water
25.5 ml aqueous solution of compound of formula (101) (100 g/l)

The temperature is kept at 98° C. for 20 min; the pH of the bath after cooling to room temperature is 5.2. The sample of PAN fabric is rinsed with cold water and subsequently dried in the air. The resulting PAN fabric contains the latent menthol. Menthol is gradually regenerated from this fabric; the velocity of menthol release is controlled by pH.

II.5 A padding bath is prepared containing 20 g/l of compound (101) and is applied at 20-25° C. with a pick-up rate of 70-80% on cotton. After drying (65 to 15 s at 70-130° C.), the resulting fabric contains the latent menthol. Menthol Is gradually regenerated from this fabric; the velocity of menthol release is controlled by pH.

II.6 The same treatment is made on cotton using compound (102) in place of (101). Subsequent to this treatment, triclosan is slowly released on the fiber, thereby ensuring good antimicrobial protection over time.

II.7 A cotton fabric is similarly treated with an aqueous formulation of compounds of formula (101) and (102). Subsequent to this treatment, menthol and triclosan are slowly released on the fibre, thereby ensuring both refreshing aromatic fragrance and good antimicrobial protection over time.

II.8 A concentrated aqueous formulation of compound (101) is sprayed on a cellulosic substrate (e.g. paper, cotton). After air-drying, the substrate containing the latent menthol releases menthol upon hydrolysis.

II.9 A concentrated aqueous formulation of compound (102) is sprayed on a cellulosic substrate (e.g. paper, cotton). After air-drying, the substrate containing the latent triclosan releases triclosan upon hydrolysis.

1. A method for the controlled release of a biologically active hydroxyl group containing substance on a substrate, which method comprises
a) reacting said hydroxyl group containing substance with a halogen-substituted aliphatic carboxylic acid halide yeilding a halogen-substituted ester,
b) reacting the ester from step a with either a diamine containing at least one tertiary amino group or a heterocyclic aromatic amine,
c) applying the thus obtained water-soluble ester to the substrate and
d) finally hydrolysing the ester on the substrate.

2. A method according to claim 1 wherein the biologically active hydroxyl group containing substance is a drug, plant protective agent, insecticide, antimicrobial, flavouring agent or cosmetics.

3. A method according to claim 2 wherein the biologically active hydroxyl group containing substance is an insecticide or an antimicrobial.

4. A method according to claim 1 wherein the substrate is selected from the group consisting of wood, plastics, paper or textile material.

5. A method according to claim 5 wherein the substrate is paper or a textile fabric.

6. A method according to claim 1 wherein the halogen-substituted aliphatic carboxylic acid halide is acetyl chloride or 4-chlorobutanoic acid chloride.

7. A method according to claim 1 wherein the diamine containing at least one tertiary amino group is of general formula R₁R₂N-A-NR₃R₄ wherein R₁ and R₂ are independently C₁-C₇ alkyl, R₃ and R₄ are independently H or C₁-C₇ alkyl and A is a C₁-C₇ linear or branched alkyl chain.

8. A method according to claim 7 wherein the diamine containing at least one tertiary amino group is 1,2-bis(dimethylamino)ethane.

9. A method according to claim 1 wherein the heterocyclic aromatic amine is an unsubstituted or substituted pyridine, bipyridyl, imidazole or oxazole.

10. A method according to claim 1 wherein the heterocyclic aromatic amine is pyridine, 4-dimethylaminopyridine, 4-methoxypyridine, 4-cyanopyridine or 4,4′-bipyridyl.

11. An aqueous solution containing the reaction product of a biologically active hydroxyl group containing substance, a halogen-substituted aliphatic carboxylic acid halide and either a diamine containing at least one tertiary amino group or a heterocyclic aromatic amine.
